(19) United States
(12) Patent Application Publication
(10) Pub. No.: US 2018/0274001 A1
(43) Pub. Date: Sep. 27, 2018

(54) NUCLEIC ACID SYNTHESIS USING DNA POLYMERASE THETA

(71) Applicant: Molecular Assemblies, Inc., San Diego, CA (US)

(72) Inventors: J. William Efcavitch, San Carlos, CA (US); Julie L. Tubbs, San Diego, CA (US)

(73) Assignee: Molecular Assemblies, Inc., San Diego, CA (US)

(21) Appl. No.: 15/926,642

(22) Filed: Mar. 20, 2018

Related U.S. Application Data
(60) Provisional application No. 62/474,426, filed on Mar. 21, 2017.

(51) Int. Cl. C12P 19/34

(52) U.S. Cl. CPC C12P 19/34; C12Y 207/07007

(57) ABSTRACT

Provided herein are methods for template-independent synthesis of oligonucleotides using a DNA polymerase. Also provided are methods for template-directed synthesis of oligonucleotides and for sequencing of nucleic acids using DNA polymerase theta and 3′-aminoalkoxy nucleotides.

### CROSS REFERENCE TO RELATED APPLICATIONS

This Application claims priority to and the benefit of U.S. Provisional Application Ser. No. 62/474,426, filed Mar. 21, 2017, the content of which is incorporated herein by reference.

### FIELD OF THE INVENTION

This invention relates to methods and compositions for synthesizing nucleic acids. The methods include template-independent synthesis of nucleic acids having a desired sequence and template-directed synthesis of nucleic acids of unknown sequences. As such, the invention provides tools and methods for medical and biological research, genetic engineering, and gene therapy.

### BACKGROUND

Most de novo nucleic acid synthesis utilizes traditional solid-phase chemical (i.e., non-enzymatic) techniques. Typical synthesis schemes involve the sequential de-protection and synthesis of sequences built from phosphoramidite reagents corresponding to natural (or non-natural) nucleic acid bases. Phosphoramidite nucleic acid synthesis is length-limited, however, in that nucleic acids greater than 200 base pairs (bp) in length experience high rates of breakage and side reactions. Additionally, phosphoramidite synthesis produces toxic byproducts, which creates disposal problems and increases cost (It is estimated that the annual demand for oligonucleotide synthesis is responsible for greater than 300,000 gallons of hazardous chemical waste, including acetonitrile, trichloroacetic acid, toluene, tetrahydrofuran, and pyridine. See LeProust et al., Nucleic Acids Res., vol. 38(8), p. 2522-2540, (2010), incorporated by reference herein in its entirety). Thus, current methods of solid-phase synthesis of nucleic acids are burdened with technical limitations, high costs, and safety hazards.

Sequencing-by-synthesis is a widely-used method for determining the sequence of existing nucleic acids. Sequencing-by-synthesis methods rely on the ability of DNA polymerases to incorporate nucleotides or nucleotide analogs into nascent DNA strands. The nucleotide analogs typically have labels that allow each nucleobase to be identified. In certain processes, nucleotide analogs with removable blocking groups are used. The blocking groups halt synthesis while a label is identified. The blocking group is then removed, allowing the addition of the next base. Traditionally, different enzymes have been used for synthesis and sequencing. In particular, polymerase enzymes useful in sequencing are not considered for use in synthesis.

### SUMMARY

The invention provides methods for template-independent, de novo synthesis of oligonucleotides using a DNA polymerase theta. The invention is based upon the unexpected result that a DNA polymerase can be used for template-independent oligonucleotide synthesis.

Methods of the invention use nucleotide analogs, such as 3′-aminoalkoxy-N4-acyl-dCTP and 3′-O-aminoalkoxy-N2-acyl-dGTP, in order to achieve stepwise synthesis of oligonucleotides. The invention utilizes DNA polymerase theta to extend single-stranded nucleic acids by incorporating nucleotide analogs having blocking moieties that prevent further elongation of the nascent strand. Removal of the blocking moiety results in conversion of the analog to a structure resembling a naturally-occurring nucleotide and allows strand elongation to resume.

In certain embodiments, methods are performed at elevated temperatures, e.g., >42° C., using a thermostable variant of DNA polymerase theta. Elevated temperatures prevent internal base-pairing of the nascent oligonucleotide, which can result in hairpin structures that hinder oligonucleotide extension. Thus, such methods increase the efficiency of oligonucleotide synthesis.

Methods and compositions of the invention offer several advantages over existing methods of nucleic acid synthesis. For solid-phase synthesis, the use of enzymatic rather than chemical synthesis enables extended synthesis runs that yield much longer oligonucleotides. The present methods also result in less waste and lower cost due to reduced complexity in the required machinery. The high-temperature methods of solid-phase synthesis eliminate the need to use nucleotide analogs that have modifications that prevent internal base-pairing. When nucleotides with modified bases are incorporated into a nascent nucleic acid, the modifications must be removed, and the removal process can leave chemical “scars” on nucleobases that distinguish them from naturally-occurring nucleobases. Consequently, the high-temperature methods facilitate simpler procedures and yield nucleic acid products that more closely resemble their natural counterparts.

In certain aspects, the invention provides methods for template-independent synthesis of an oligonucleotide. Preferred methods include combining an initiator nucleic acid linked to a solid support, a nucleotide analog, and a DNA polymerase theta, or analog thereof, in an aqueous solution, causing the DNA polymerase to incorporate the nucleotide analog into the nucleic acid. The nucleotide analog includes a removable blocking moiety that prevents the DNA polymerase from attaching additional nucleotides or nucleotide analogs to the nucleic acid. Upon removal of the blocking moiety from the nucleotide analog, however, the DNA polymerase is able to attach additional nucleotides or nucleotide analogs to the nucleic acid. The aqueous solution may include Mn²⁺.

The DNA polymerase polypeptide may be any polypeptide that has DNA polymerase activity, such as the catalytic subunit of the DNA polymerase complex. Preferably, the DNA polymerase is DNA polymerase theta. The DNA polymerase theta polypeptide may be derived from any multicellular eukaryote, such as a human, other animal, insect, nematode, fungus, etc. The DNA polymerase theta polypeptide may have an amino acid sequence corresponding to a full-length gene product or a portion thereof, such as the polymerase domain. The DNA polymerase theta polypeptide may have an amino acid sequence identical to a naturally-occurring gene product, or it may have an amino acid sequence with alterations, such as insertions, deletions, or substitutions.

The oligonucleotide may be DNA, or a hybrid of DNA & RNA. The initiator nucleic acid bound to a solid support may be single-stranded nucleic acid. Preferably, the initiator nucleic acid is single-stranded DNA. For RNA synthesis, the preferred embodiment of the initiator is DNA. If a hybrid DNA-RNA oligonucleotide is used, the preferred embodiment comprises the DNA portion at the 5′-end of the initiator. The initiator nucleic acid may have a modified nucleotide at its 3′ terminus that joins with a nucleotide analog to form a covalent bond that is cleavable under conditions that do not break phosphodiester bonds between adjacent nucleotides in the oligonucleotide.

The nucleotide analogs may be analogs of deoxyribonucleotide triphosphates (dNTPs, e.g., dATP, dCTP, dGTP, and dTTP) or ribonucleotide triphosphates (rNTPs, e.g., rATP, rCTP, rGTP, rUTP) that are the natural substrates for synthesis of nucleic acids. Thus, the nucleotide analogs may include a ribose component, a base component, and a phosphate component.

The removable blocking moiety of the nucleotide analog may be linked via one or more of the carbon atoms at the 2′, 3′, and 4′ positions of the ribose ring. Preferably, the removable blocking moiety is linked via the 3′ position in the ribose ring. The removable blocking moiety may be a 3′-aminoalkoxy group or a 3′-O-azidomethyl group. Alternatively, the removable blocking moiety may be linked via the base of the nucleotide analog. For example, the removable blocking moiety may be linked via N4 of cytosine, N3 of thymine, 04 of thymine, N2 of guanine, N3 of guanine, N6 of adenine, N3 of uracil, or 04 of uracil. The nucleotide analogs may be 3′-aminoalkoxy dNTPs or 3′-aminoalkoxy rNTPs. For example, the nucleotide analogs may be 3′-aminoalkoxy-N4-acyl-dCTP, 3′-aminoalkoxy-N4-acyl-rCTP, 3′-aminoalkoxy-N2-acyl-dGTP, or 3′-aminoalkoxy-N2-acyl-rGTP.

One or more of the nucleotide analogs may include a removable moiety that inhibits base-pairing between the nucleotide analog and other nucleotides or nucleotide analogs. The base-pair-inhibiting moiety may be the same as the blocking moiety, or the two may be different. Preferably, the blocking moiety and base-pair-inhibiting moiety are different. The base-pair-inhibiting moiety and the blocking moiety may be removable under the same conditions, or they may be removable under different conditions. Preferably, the base-pair-inhibiting moiety remains attached to the nucleotide analog under conditions that result in removal of the blocking moiety. The removable base-pair-inhibiting moiety may be linked via N6 of adenine, N2 of guanine, or N4 of cytosine.

One or more of the nucleotide analogs may include a removable moiety that increases the rate of incorporation of the nucleotide analog comprising a removable blocking group. For example, modifications at N6 of adenine or N2 of guanine can enhance the incorporation rate of nucleotide analogs modified at the 3′-OH. The nucleotide analogs may be 3′-aminoalkoxy dNTPs or 3′-aminoalkoxy rNTPs. For example, the nucleotide analogs may be 3′-aminoalkoxy-N6-arylacyl-dATP, 3′-aminoalkoxy-N6-amidine-dATP, 3′-aminoalkoxy-N2-arylacyl-dGTP, or 3′-aminoalkoxy-N2-arylacyl-rGTP. Such removable modifications may serve the dual purpose of increasing the rate of enzymatic incorporation rate of the nucleotide analog and inhibiting base-pairing between the nucleotide analog and other nucleotides or nucleotide analogs. The rate-enhancing moiety and the blocking moiety may be removable under the same conditions, or they may be removable under different conditions. Preferably, the rate-enhancing moiety remains attached to the nucleotide analog under conditions that result in removal of the blocking moiety. Preferably, the rate-enhancing and base-pair inhibiting moiety are removable under the same conditions.

One or more of the nucleotide analogs may include a removable label that allows identification of the base component of the nucleotide analog. The label may be a fluorescent label. A set of nucleotide analogs may include bases that correspond to the four naturally-occurring bases in dNTPs (A, C, T, and G) or rNTPs (A, C, T, and U). At least one nucleotide analog in a set may contain a unique label. Preferably, each of the four the nucleotide analogs in a set contains a unique label. The removable label may be linked to the nucleotide analog via one or more of the carbon atoms at the 2′, 3′, and 4′ positions of the ribose ring or via an atom in the base of the nucleotide analog. The label and the blocking moiety may be removable under the same conditions, or they may be removable under different conditions. For example, the label may be removable under conditions that result in conversion of the 3′-aminoalkoxy group to a 3′-OH group, or the label may be removable under different conditions.

Methods of the invention may include additional steps. For example, methods may include one or more of the following: removing the removable blocking moiety from the nucleotide analog; removing the initiator nucleic acid from the solid support; removing the base-pair-inhibiting moiety from the nucleotide analog; removing the rate-enhancing moiety from the nucleotide analog; cleaving the covalent bond between the nucleotide analog and terminal nucleotide of the initiator nucleic acid; and digesting the initiator nucleic acid with DNase.

In certain aspects, the invention provides methods for template-independent synthesis of an oligonucleotide using a thermostable DNA polymerase, preferably a thermostable polymerase theta. Those methods entail combining an initiator nucleic acid linked to a solid support, a nucleotide analog, and a thermostable DNA polymerase polypeptide in an aqueous solution, causing the thermostable DNA polymerase polypeptide to incorporate the nucleotide analog into the nucleic acid. The nucleotide analog includes a removable blocking moiety that prevents the thermostable DNA polymerase polypeptide from attaching additional nucleotides or nucleotide analogs to the nucleic acid. Upon removal of the blocking moiety from the nucleotide analog, however, the thermostable DNA polymerase polypeptide is able to attach additional nucleotides or nucleotide analogs to the nucleic acid. Preferably, the aqueous solution includes Mn²⁺.

The thermostable DNA polymerase polypeptide may be any polypeptide that has DNA polymerase activity at elevated temperatures, for example, >42° C. The thermostable DNA polymerase polypeptide may be an engineered polypeptide that includes amino acid sequences from two or more different DNA polymerases, such as different A-family DNA polymerase. For example, the DNA polymerase may include a catalytic region from a thermostable DNA polymerase, such Taq, and one or more loop domains from a DNA polymerase theta.

The reagents may be combined at an elevated temperature when a thermostable DNA polymerase is used. For example, the reagents may be combined at >42° C. The elevated temperature may be selected to prevent formation of hairpin or other secondary structures of the nascent oligonucleotide due to base-pairing between self-complementary regions during synthesis. The elevated temperatures may obviate the need for modifications that prevent the nucleotide analogs from forming base pairs. Thus, the nucleotide analogs may be free of base-pair-inhibiting moieties.

The features described above in relation to methods for template-independent synthesis of an oligonucleotide using a DNA polymerase, such as DNA polymerase theta, may be incorporated as relevant to methods that involve use of a thermostable DNA polymerase.

In other aspects, the invention provides methods for template-directed synthesis of an oligonucleotide. The methods include combining a nucleic acid template, a nucleic acid primer, a 3′-aminoalkoxy nucleotide analog, and DNA polymerase theta in an aqueous solution, causing DNA polymerase theta to attach the 3′-aminoalkoxy nucleotide analog to the primer. The primer anneals to a sequence in the template, and the nucleotide analog is complementary to the nucleotide immediately 5′ to the primer-binding sequence in the template. The 3′-aminoalkoxy group in the nucleotide analog prevents DNA polymerase theta from attaching additional nucleotides or nucleotide analogs to the nascent oligonucleotide. Upon conversion of the 3′-aminoalkoxy group to a 3′-OH group, however, DNA polymerase theta is able to attach additional nucleotides or nucleotide analogs to the nascent oligonucleotide. The aqueous solution may include Mn²⁺ or Mn²⁺.

The features described above in relation to methods for template-independent synthesis of an oligonucleotide may be incorporated as relevant to methods for template-directed synthesis of an oligonucleotide.

In other aspects, the invention provides methods for determining the nucleotide sequence of a nucleic acid molecule. The methods include combining the following in an aqueous solution: a nucleic acid template that includes a portion of the nucleic acid molecule to be sequenced; a nucleic acid primer complementary to a nucleotide sequence in the template; a 3′-aminoalkoxy nucleotide analog that includes a removable label linked to a base of the nucleotide analog and that is complementary to the nucleotide immediately 5′ to the primer-binding sequence in the template; and DNA polymerase theta. This step results in formation of a covalent bond between the nucleotide analog and the terminal nucleotide of the nucleic acid primer. The methods further include the steps of identifying the nucleotide analog in the sequence complementary to the nucleic acid template, removing the removable label from the base of the nucleotide analog, and converting the 3′-aminoalkoxy group of the nucleotide analog to a 3′-OH group. Identification of the nucleotide analog determines at least a portion of the sequence of the nucleic acid molecule. Preferably, the aqueous solution includes Mg²⁺.

Certain methods of the invention are also useful in template-dependent sequencing of oligonucleotides using DNA polymerase theta and 3′-aminoalkoxy nucleotide analogs in the absence of MN⁺. This is based upon the discovery that 3′-aminoalkoxy-dNTPs are incorporated by polymerase theta in the presence of Mn²⁺. It is thus proposed herein that the same nucleotide analogs are incorporated in DNA sequencing using polymerase theta in the presence of Mg²⁺ using the natural template-dependent mechanism of polymerase theta when not in the presence of Mn2+. Consequently, oligonucleotide synthesis cycles between steps of addition of a 3′-aminoalkoxy nucleotide analog and conversion of its 3′-aminoalkoxy group. In certain embodiments, the 3′-aminoalkoxy nucleotide analogs include removable labels, such as fluorescent labels, that signify the base component of the analog. In such methods, the label can be detected and subsequently removed during each cycle. Therefore, these methods are useful for determining the nucleotide sequence of a nucleic acid.

The features described above in relation to methods for template-independent synthesis of an oligonucleotide may be incorporated as relevant to methods for determining the nucleotide sequence of a nucleic acid molecule.

### BRIEF DESCRIPTION OF THE DRAWINGS

FIG. 1 illustrates a method for template-independent nucleic acid synthesis according to an embodiment of the invention.

FIG. 2 shows the removal of a removable blocking moiety from a nascent oligonucleotide.

FIG. 3 shows the removal of removable base-pair-inhibiting moieties from a nascent oligonucleotide.

FIG. 4 illustrates a method for template-independent nucleic acid synthesis according to an embodiment of the invention.

FIG. 5 illustrates a method for template-directed nucleic acid synthesis of according to an embodiment of the invention.

FIG. 6 illustrates a method for determining the nucleotide sequence of a nucleic acid according to an embodiment of the invention.

### DETAILED DESCRIPTION

The invention generally relates to compositions and methods for synthesis of nucleic acids. The invention provides methods for template-independent synthesis of nucleic acids using DNA polymerase theta. In the presence of Mn²⁺, DNA polymerase theta incorporates nucleotide analogs into a nucleic acid primer in the absence of a template. The nucleotide analogs include reversibly-attached blocking moieties that prevent attachment of additional nucleotides or nucleotide analogs, so each round of nucleotide addition is followed by removal of the blocking moiety. Consequently, the sequence of the oligonucleotide is specified by providing a selected nucleotide analog during each cycle of nucleotide addition.

The invention also provides methods for template-directed synthesis of nucleic acids using DNA polymerase theta and 3′-aminoalkoxy-modified nucleotide triphosphates (NTPs). In the presence of Mg²⁺, DNA polymerase theta incorporates 3′-aminoalkoxy NTPs into a nucleic acid primer. Because 3′-aminoalkoxy NTPs lack a free 3′-OH group, incorporation of a 3′-aminoalkoxy nucleotide into the nascent oligonucleotide blocks strand elongation. However, conversion of the 3′-aminoalkoxy group to a 3′-OH group allows DNA polymerase theta to resume strand elongation. Consequently, template-directed synthesis proceeds by alternating steps of addition of 3′-aminoalkoxy nucleotides and conversion of the 3′ substituent on the ribose ring. In particular application of these methods, nucleic acid synthesis is performed using a mixture of 3′-aminoalkoxy NTPs that mirror the four naturally-occurring nucleotide substrates for DNA or RNA synthesis and that each have unique, removable labels, e.g., fluorescent labels. Because only one labeled, 3′-aminoalkoxy nucleotide analog is added during each cycle of strand synthesis, the identity of the newly-added nucleotide analog can be determined from its label. Thus, due to the requirement of base complementarity between the nascent and template strands, the nucleotide sequence of the template nucleic acid can be determined.

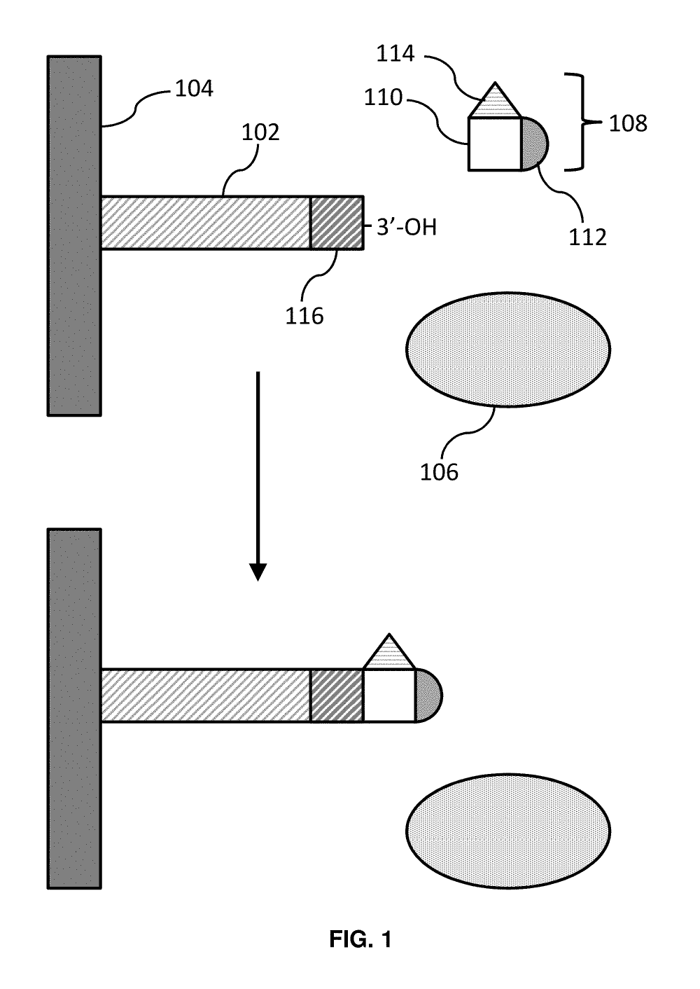

FIG. 1 illustrates a method for template-independent nucleic acid synthesis according to an embodiment of the invention. An initiator nucleic acid 102 bound to a solid substrate 104 is combined with a DNA polymerase 106 and a nucleotide analog 108 in an aqueous solution. The nucleotide analog 108 includes a nucleotide component 110 and removable blocking moiety 112. The nucleotide analog may also include a removable base-pair-inhibiting and/or rate-enhancing moiety 114, as shown in FIG. 1. The initiator nucleic acid 102 has a terminal nucleotide 116 with a free 3′-OH group. The DNA polymerase 106 catalyzes the formation of a covalent bond between the nucleotide analog 108 and the terminal nucleotide 116 of the initiator nucleic acid 102. The presence of the blocking moiety 112 on the nucleotide analog 108 prevents strand elongation by blocking the DNA polymerase 106 from attaching additional nucleotides (not shown) or nucleotide analogs 108.

DNA polymerases have been categorized in seven evolutionary families based on their amino acid sequences: A, B, C, D, X, Y, and RT. The families of DNA polymerases appear to be unrelated, i.e., members of one family are not homologous to members of any other family. A DNA polymerase is determined to be a member of given family by its homology to a prototypical member of that family. For example, members of family A are homologous to E. coli DNA polymerase I; members of family B are homologous to E. coli DNA polymerase II; members of family C are homologous to E. coli DNA polymerase III; members of family D are homologous to Pyrococcus furiosus DNA polymerase; members of family X are homologous to eukaryotic DNA polymerase beta; members of family Y are homologous to eukaryotic RAD30; and members of family RT are homologous to reverse transcriptase. For many years, the only DNA polymerases known to perform template-independent DNA synthesis were members of family X, such as terminal deoxynucleotidyl transferase (TdT), DNA polymerase mu, and nucleotidyltransferases. Recent reports, however, have revealed that DNA polymerase theta, a member of family A, is capable of template-independent DNA polymerase activity.

In humans, DNA polymerase theta is encoded by the POLQ gene, which encodes a polypeptide of 2590 amino acids (SEQ ID NO:1). DNA polymerase theta includes a helicase at its amino terminus (residues 1-894; SEQ ID NO:2), an A-family polymerase at its carboxy terminus (residues 1792-2590; SEQ ID NO:4), and a large central portion of unknown function (residues 895-1791; SEQ ID NO:3) (see Black, S. J. et al., DNA Polymerase θ: A Unique Multifunctional End-Joining Machine, Genes 7:67 (2016) for more details). The polymerase domain of DNA polymerase theta has a similar sequence and structure to other family A polymerases, but it also includes conserved loop domains corresponding to residues 2149-2170 (SEQ ID NO:5), 2264-2315 (SEQ ID NO:6), and 2497-2529 (SEQ ID NO:7) of the human theta polypeptide.

TABLE 1
Amino acid sequences of human DNA polymerase theta

| SEQ ID NO | Description |
|---|---|
| 1 | full length (residues 1-2590) |
| 2 | helicase domain (residues 1-894) |
| 3 | central domain (residues 895-1791) |
| 4 | polymerase domain (residues 1792-2590) |
| 5 | loop 1 in polymerase domain (2149-2170) |
| 6 | loop 2 in polymerase domain (2264-2315) |
| 7 | loop 3 in polymerase domain (2497-2529) |

DNA polymerase theta has different functional properties from other family A members, such as E. coli DNA pol I (Klenow fragment), Taq polymerase, T7 DNA and RNA polymerases, and Pol gamma. Compared to other A-family DNA polymerases, DNA polymerase theta synthesizes new strands with low-fidelity polymerase and is efficient at extending mismatched primer termini. DNA polymerase theta is also highly efficient at translesion synthesis, i.e., synthesizing a new strand across lesions, such as abasic sites and thymine glycols, in the template strand. It is also the only A-family member known to have template-independent polymerase activity. The loop domains in the polymerase domain of theta are critical for these atypical functions: loops 2 and 3 are necessary for translesion activity, loop 2 is required for terminal transferase (i.e., template-independent) activity, and loop 1 promotes processivity of the enzyme.

The present invention is based in part on the finding that the unmodified polymerase domain of DNA polymerase theta can use reversible terminator nucleotide analogs as substrates for template-independent oligonucleotide synthesis. Prior to the invention described herein, there have been no reports of template-independent nucleic acid synthesis by DNA polymerase theta using nucleotide analogs as substrates. The inventors have found, however, that DNA polymerase theta efficiently incorporates nucleotide analogs that have 3′ modifications. For example, 3′-O-azidomethyl dCTP, 3′-O-cyanoethyl dATP or 3′-aminoalkoxy dNTPs are readily incorporated into oligonucleotides in a template-independent manner by DNA polymerase theta. In contrast, these analogs are incorporated with slow kinetics by TdT, the enzyme used most widely for template-independent DNA synthesis in research applications. Thus, in preferred embodiments, DNA polymerase 106 is a DNA polymerase theta polypeptide.

As used herein, a “DNA polymerase theta polypeptide” refers to any polypeptide that has one or more amino acid sequences derived from a DNA polymerase theta of any organism and that has DNA polymerase activity. The DNA polymerase theta polypeptide may have one or more amino acid sequences identical to those in a naturally-occurring DNA polymerase theta. The DNA polymerase theta polypeptide may have one or more amino acid sequences that have alterations to amino acid sequences from a naturally-occurring DNA polymerase theta. The alterations may include amino acid substitutions, insertions, deletions, or modifications. The DNA polymerase polypeptide may include an amino acid sequence that is at least 70%, at least 75%, at least 80%, at least 85%, at least 90%, at least 95%, at least 96%, at least 97%, at least 98%, or at least 99% identical to SEQ ID NO:4.

The inventors have discovered that Mn²⁺ promotes DNA polymerase theta's ability to use reversible terminator nucleotide analogs as substrates for template-independent oligonucleotide synthesis. Thus, in preferred embodiments of the invention, the aqueous solution contains Mn²⁺. The Mn²⁺ concentration may be about 0.05 mM, about 0.1 mM, about 0.2 mM, about 0.5 mM, about 1 mM, about 2 mM, about 5 mM, or about 10 mM.

The invention also contemplates modified or engineered forms of DNA polymerase theta that use nucleotide analogs with higher efficiency for template-independent oligonucleotide synthesis. Modifying one or more amino acid residues in the active site of the enzyme can increase the efficiency of incorporation of 3′-blocked nucleotide analogs into a support-bound initiator. Protein engineering or protein evolution can also be used to modify the enzyme to optimize the use of analogs of each of the four different nucleobases or even different nucleobase analogs in an analog-specific manner. Nucleotide-specific or nucleotide-analog-specific enzyme variants could be engineered to possess desirable biochemical attributes like reduced Kₘ or enhanced addition rate, which would further reduce the cost of the synthesis of desired oligonucleotides.

Another normally template dependent DNA polymerase that also shows the ability to incorporate 3′-O-modified nucleotides in the presence of Mn²⁺ in a template-independent fashion is Therminator from Thermococcus sp. 9° N.

In another embodiment, protein engineering or protein evolution is used to modify DNA polymerase theta to remain tightly bound to the nascent strand after each single nucleotide incorporation, thus preventing any subsequent incorporation until such time as the polymerase/transferase is released from the strand by use of a releasing reagent/condition. Such modifications would be selected to allow the use of natural, unmodified NTPs, rather than NTPs that have blocking moieties, as substrates. Releasing reagents could be high salt buffers, denaturants, etc. Releasing conditions could be high temperature, agitation, etc. Other means of accomplishing the goal of a post-incorporation, tight-binding polymerase enzyme could include mutations to the residues responsible for binding the three phosphates of the initiator strand.

The initiator nucleic acid 102 serves as a binding site for the DNA polymerase 106. The initiator nucleic acid 102 may be RNA or DNA and may be single-stranded or partially single-stranded. Preferably, the initiator nucleic acid 102 is single-stranded DNA. It is hypothesized that stepwise oligonucleotide synthesis using nucleotide analogs with blocking moieties causes the DNA polymerase to release the nascent oligonucleotide, and the use of a single-stranded DNA initiator promotes re-binding of the DNA polymerase during each cycle. The initiator nucleic acid 102 may have a user-defined sequence or may be a universal initiator, such as a homopolymer, from which the user-defined, single-stranded product is removed. The initiator nucleic acid 102 may be recyclable on the solid support and may have a sequence that allows cleavage of the synthesized oligonucleotide from the initiator nucleic acid 102, for example, by a restriction endonuclease. The initiator nucleic acid 102 may be any length that provides a sufficient binding site for the DNA polymerase 106. At the 3′ end of the initiator nucleic acid 102 is a terminal nucleotide 116. Preferably, the terminal nucleotide 116 has a free 3′-OH group to which the DNA polymerase 106 can attach the nucleotide analog 108 via a phosphodiester bond. The terminal nucleotide 116 may also have a non-naturally-occurring 3′ group that allows the formation of a cleavable, non-phosphodiester bond with the oligonucleotide, which can be cleaved upon completion of oligonucleotide synthesis to yield the oligonucleotide having the specified sequence with no additional 5′ nucleotides.

The solid support 104 may be any solid support compatible with nucleic acid synthesis. Solid supports suitable for use with the methods of the invention may include glass and silica supports, including beads, slides, pegs, or wells. In some embodiments, the support may be tethered to another structure, such as a polymer well plate or pipette tip. In some embodiments, the solid support may have additional magnetic properties, thus allowing the support to be manipulated or removed from a location using magnets. In other embodiments, the solid support may be a silica coated polymer, thereby allowing the formation of a variety of structural shapes that lend themselves to automated processing.

The oligonucleotide synthesized by the methods may be DNA, RNA, or a DNA/RNA hybrid. The oligonucleotide may have a length of up to 5000 nt.

The nucleotide analog 108 is an analog of a naturally-occurring nucleotide triphosphate. As such, the nucleotide analog 108 includes a ribose ring, a base attached to the 1′ carbon in the ribose ring, and a phosphate component attached to the 5′ carbon of the ribose ring. To promote oligonucleotide synthesis, the nucleotide analog is a nucleotide triphosphate. To synthesize a DNA oligonucleotide, analogs of deoxyribonucleotide triphosphates (dNTPs), i.e., nucleotide triphosphates having no —OH group at the 2′ position in the ribose ring, are used. Preferably, each dNTP has one of the four bases (adenine, cytosine, guanine, and thymine) found in naturally-occurring DNA. To synthesize a RNA oligonucleotide, analogs of ribonucleotide triphosphates (rNTPs), i.e., nucleotide triphosphates having a —OH group at the 2′ position in the ribose ring, are used. Preferably, the each rNTP has one of the four bases (adenine, cytosine, guanine, and uracil) found in naturally-occurring RNA.

In the illustration shown in FIG. 1, the nucleotide analog 108 includes a nucleotide 110 component attached to a removable blocking moiety 112. The nucleotide analog 108 can be used as a substrate by the DNA polymerase 106 for elongation of the initiator nucleic acid 102 by formation of a covalent bond, e.g., a phosphodiester bond, with the free 3′-OH group of the terminal nucleotide 116 of the initiator nucleic acid 102. Upon attachment of the nucleotide analog 108 to the initiator nucleic acid 102, the removable blocking moiety 112 prevents further elongation of the nascent oligonucleotide by the DNA polymerase 106.

While synthetic pathways for “natural” nucleotides, such as DNA and RNA, are described in the context of the common nucleic acid bases, e.g., adenine (A), guanine (G), cytosine (C), thymine (T), and uracil (U), it is to be understood that the methods of the invention can be applied to so-called “non-natural” nucleotides, including nucleotides incorporating universal bases such as 3-nitropyrrole 2′-deoxynucloside and 5-nitroindole 2′-deoxynucleoside, alpha phosphorothiolate, phosphorothioate nucleotide triphosphates, or purine or pyrimidine conjugates that have other desirable properties, such as fluorescence. Other examples of purine and pyrimidine bases include pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine,6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine. Other hydrophobic non-natural base analogs which may be useful are 2-((2R,4R,5R)-tetrahydro-4-hydroxy-5-(hydroxymethyl)furan-2-yl)-6-methylisoquinoline-1(2H)-thione (5SICS), 1,4-Anhydro-2-deoxy-1-C-(3-methoxy-2-naphthalenyl)-(1R)-D-erythro-pentitol (dNaM), ((2R,3R,5R)-5-(8-amino-3H-imidazo[4,5-g]quinazolin-3-yl)-3-hydroxytetrahydrofuran-2-yl)methyl (dxA), ((2R,3R,5R)-5-(4-amino-6-methyl-2-oxo-1,2-dihydroquinazolin-8-yl)-3-hydroxytetrahydrofuran-2-yl)methyl (dxC), ((2R,3R,5R)-5-(6-amino-8-oxo-7,8-dihydro-3H-imidazo[4,5-g]quinazolin-3-yl)-3-hydroxytetrahydrofuran-2-yl)methyl (dxG), ((2R,3R,5R)-3-hydroxy-5-(6-methyl-2,4-dioxo-1,2,3,4-tetrahydroquinazolin-8-yl)tetrahydrofuran-2-yl)methyl (dxT). In some instances, it may be useful to produce nucleotide sequences having unreactive, but approximately equivalent bases, i.e., bases that do not react with other proteins, i.e., transcriptases, thus allowing the influence of sequence information to be decoupled from the structural effects of the bases.

The removable blocking moiety 112 may be any moiety that blocks attachment of additional nucleotides or nucleotide analogs to the nascent oligonucleotide. The removable blocking moiety 112 may be a substituent at the 3′ position of the ribose ring that prevents formation of a phosphodiester bond at this position, as described in detail below. The removable blocking moiety 112 may be a substituent at the 2′ and/or 4′ positions of the ribose ring that sterically interferes with formation of a phosphodiester bond at the 3′ position. The removable blocking moiety 112 may be linked to the base of the nucleotide analog and may prevent strand elongation by sterically interfering with the DNA polymerase 106, as described in detail below.

Examples of blocking moieties at the 3′ position of the ribose ring include, but are not limited to, the 3′-O-allyl, 3′-O-azidomethyl (3′-OCH₂N₃), 3′-aminoalkoxyl (3′-ONH₂), and 3′-OCH₂CN blocking groups. Overall, the choice of the 3′-O-blocking group will be influenced by the blocking group with the mildest removal conditions, preferably aqueous, and in the shortest period of time. 3′-O-blocking groups that are the suitable for use with this invention are described in WO 2003/048387; WO 2004/018497; WO 1996/023807; WO 2008/037568; Hutter D, et al. Nucleosides Nucleotides Nucleic Acids, 2010, 29(11): 879-95; and Knapp et al., Chem. Eur. J., 2011, 17:2903, all of which are incorporated by reference in their entireties.

Thus, a variety of 3′-O-modified dNTPs and rNTPs may be used for oligonucleotide synthesis. In some embodiments, the preferred removable 3′-O-blocking moiety is a 3′-O-amino (e.g., 3′-ONH₂), a 3′-O-allyl, a 4′-O-cyanoethyl, or a 3′-O-azidomethyl. In other embodiments, the removable 3′-O-blocking moiety is selected from the group consisting of O-phenoxyacetyl; O-methoxyacetyl; O-acetyl; O-(p-toluene)-sulfonate; O-phosphate; O-nitrate; O-[4-methoxy]-tetrahydrothiopyranyl; O-tetrahydrothiopyranyl; O-[5-methyl]-tetrahydrofuranyl; O-[2-methyl,4-methoxy]-tetrahydropyranyl; O-[5-methyl]-tetrahydropyranyl; and O-tetrahydrothiofuranyl (see U.S. Pat. No. 8,133,669). In other embodiments the removable blocking moiety is selected from the group consisting of esters, ethers, carbonitriles, phosphates, carbonates, carbamates, hydroxylamine, borates, nitrates, sugars, phosphoramide, phosphoramidates, phenylsulfenates, sulfates, sulfones and amino acids (see Metzker M L et al. Nuc Acids Res. 1994; 22(20):4259-67, U.S. Pat. Nos. 5,763,594, 6,232,465, 7,414,116; and 7,279,563, all of which are incorporated by reference in their entireties).

Nucleotide analogs that have blocking moieties linked to a base may have the formula NTP-linker-inhibitor for synthesis of nucleic acids in an aqueous environment, such as those described in U.S. Pat. No. 8,808,989, which is incorporated herein by reference. With respect to the analogs of the form NTP-linker-inhibitor, NTP can be any nucleotide triphosphate, such as adenosine triphosphate (ATP), guanosine triphosphate (GTP), cytidine triphosphate (CTP), thymidine triphosphate (TTP), uridine triphosphate (UTP), nucleotide triphosphates, deoxyadenosine triphosphate (dATP), deoxyguanosine triphosphate (dGTP), deoxycytidine triphosphate (dCTP), deoxythymidine triphosphate (dTTP), or deoxyuridine triphosphate (dUTP).

The linker can be any molecular moiety that links the inhibitor to the NTP and can be cleaved, e.g., chemically cleaved, electrochemically cleaved, enzymatically cleaved, or photolytically cleaved. For example, the linkers can be cleaved by adjusting the pH of the surrounding environment. The linkers may also be cleaved by an enzyme that is activated at a given temperature, but inactivated at another temperature. In some embodiments, the linkers include disulfide bonds.

The linker can be attached, for example, at the N4 of cytosine, the N3 or O4 of thymine, the N2 or 06 of guanine, and the N6 of adenine, or the N3 or O4 of uracil because attachment at a carbon results in the presence of a residual scar after removal of the polymerase-inhibiting group. The linker is typically on the order of at least about 10 Angstroms long, e.g., at least about 20 Angstroms long, e.g., at least about 25 Angstroms long, thus allowing the inhibitor to be far enough from the pyridine or pyrimidine to allow the enzyme to bind the NTP to the oligonucleotide chain via the attached sugar backbone. In some embodiments, the cleavable linkers are self-cyclizing in that they form a ring molecule that is particularly non-reactive toward the growing nucleotide chain.

The nucleotide analogs can include any moiety linked to the NTP that inhibits the coupling of subsequent nucleotides by the enzyme. The inhibitory group can be a charged group, such as a charged amino acid, or the inhibitory group can be a group that becomes charged depending upon the ambient conditions. In some embodiments, the inhibitor may include a moiety that is negatively charged or capable of becoming a negatively charged. In other embodiments, the inhibitor group is positively charged or capable of becoming positively charged. In some other embodiments, the inhibitor is an amino acid or an amino acid analog. The inhibitor may be a peptide of 2 to 20 units of amino acids or analogs, a peptide of 2 to 10 units of amino acids or analogs, a peptide of 3 to 7 units of amino acids or analogs, a peptide of 3 to 5 units of amino acids or analogs. In some embodiments, the inhibitor includes a group selected from the group consisting of Glu, Asp, Arg, His, and Lys, and a combination thereof (e.g., Arg, Arg-Arg, Asp, Asp-Asp, Asp, Glu, Glu-Glu, Asp-Glu-Asp, Asp-Asp-Glu or AspAspAspAsp, etc.). Peptides or groups may be combinations of the same or different amino acids or analogs. The inhibitory group may also include a group that reacts with residues in the active site of the enzyme thus interfering with the coupling of subsequent nucleotides by the enzyme.

The inhibitor coupled to the nucleotide analog prevents the DNA polymerase from releasing the oligonucleotide or prevents other analogs from being incorporated into the growing chain. In some embodiments, the inhibitor includes single amino acids or dipeptides, like -(Asp)₂. However the size and charge on the moiety can be adjusted, as needed, based upon experimentally determined rates of first nucleotide incorporation and second nucleotide incorporation. Thus, other embodiments may use more or different charged amino acids or other biocompatible charged molecule.

Other modifications to the base portion of a dNTP analog that may be efficacious at preventing the addition of more than one nucleotide by polymerase theta are N4-isobutryl-dCTP, N4-benzoyl-dCTP, and N3-allyl-dTTP. During oligonucleotide synthesis, nucleotide sequences in one portion of the strand may anneal with complementary nucleotide sequences in other portions of the strand may anneal with each other. The resulting hairpin structure may hinder the rate and/or yield of enzymatic extension, reducing the efficiency of synthesis of the desired full length product. Consequently, to prevent formation of hairpin structures, the nucleotide analog 108 may also have a removable moiety that inhibits formation of base pairs. Preferably, the base-pair-inhibiting moiety remains on the nascent oligonucleotide chain during repetitive cycles of reversible terminator addition, thus preventing hairpin formation and insuring high yield enzymatic synthesis of long oligonucleotides.

The base-pair-inhibiting moiety may be any removable substituent that obviates base-pairing and hairpin formation. The base-pair-inhibiting moiety may be attached the base of the nucleotide analog. Preferably, base-pair-inhibiting moiety is attached to an exocyclic amine of the nucleotide analog, such as N6 of adenine, N4 of cytosine, or N2 of guanine. The base-pair-inhibiting moiety may be an acyl group. Thus, exemplary nucleotide analogs include 3′-aminoalkoxy-N4-acyl-dCTP and 3′-aminoalkoxy-N2-acyl-dGTP.

It is desirable to have the base-pair-inhibiting moiety remain bound to the nucleotide analog until strand elongation is complete. Because strand elongation entails cycles of nucleotide addition followed by deblocking, it is advantageous that the base-pair-inhibiting moieties remain attached to the nucleotide analogs under conditions that remove the blocking moiety.

The inventors have discovered that certain substituents on the bases of nucleotide analogs enhance the rate at which DNA polymerase theta incorporates the nucleotide analogs into a nascent oligonucleotide. For example, modifications, such as aromatic amides or amidines, at N6 of adenine or N2 of guanine in nucleotide analogs modified at the 3′-OH can enhance the rate of incorporation by DNA polymerase theta. Consequently, to promote more rapid oligonucleotide synthesis, the nucleotide analog 108 may also have a removable moiety that enhances the rate of incorporate of the nucleotide analog by DNA polymerase theta. Preferably, the rate-enhancing moiety remains attached to the nucleotide analog under conditions that result in removal of the blocking moiety. The nucleotide analogs that have rate-enhancing moieties may be 3′-aminoalkoxy dNTPs or 3′-aminoalkoxy rNTPs. For example, the nucleotide analogs may be 3′-aminoalkoxy-N6-arylacyl-dATP, 3′-aminoalkoxy-N6-amidine-dATP, 3′-aminoalkoxy-N2-arylacyl-dGTP, or 3′-aminoalkoxy-N2-arylacyl-rGTP.

A single base substituent may serve the dual purpose of increasing the rate of enzymatic incorporation of the nucleotide analog and inhibiting base-pairing between the nucleotide analog and other nucleotides or nucleotide analogs. Thus, the base-pair-inhibiting and/or rate-enhancing moiety 114 may be a single moiety that serves both functions, as illustrated in FIG. 1. Alternatively, base-pair-inhibiting moiety and rate-enhancing moiety may be separate moieties. If the base-pair-inhibiting moiety and rate-enhancing moieties are distinct, they may be removable under the same conditions or under different conditions.

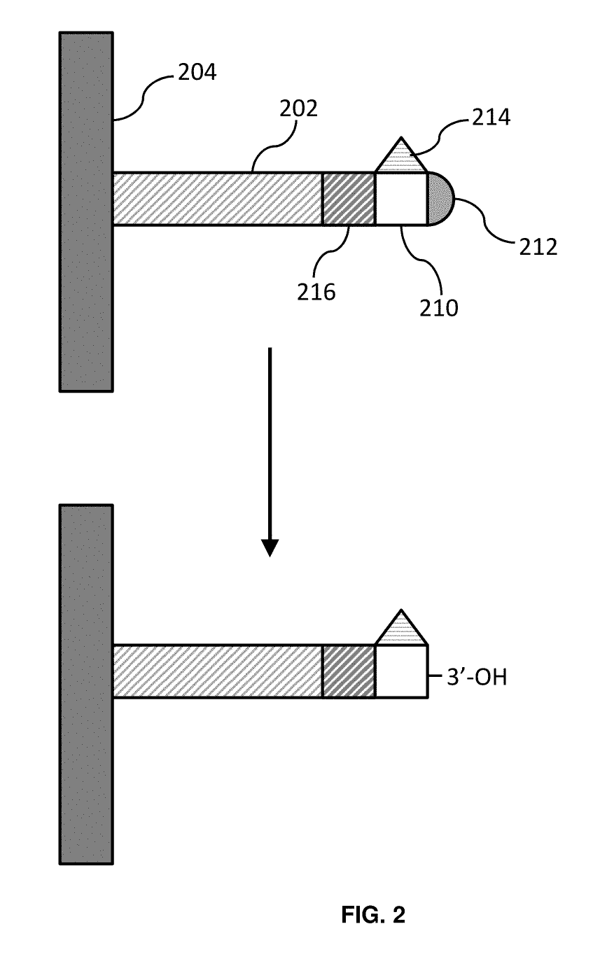

FIG. 2 shows the removal of a removable blocking moiety 212 from a nascent oligonucleotide. The nascent oligonucleotide is the product of the reaction illustrated in FIG. 1. The following components shown in FIG. 2 are described above in relation to FIG. 1: a solid support 204; an initiator nucleic acid 202, which includes a terminal nucleotide 216, attached to the solid support; and a nucleotide analog (not labeled) that is attached to the terminal nucleotide 216 and that includes a nucleotide component 210, removable blocking moiety 212, and a base-pair-inhibiting and/or rate-enhancing moiety 214. In the step illustrated in FIG. 2, the removable blocking moiety 212 is removed from the nascent oligonucleotide. Upon removal of the blocking moiety 212, the 3′-OH group of the nucleotide analog is available to react with another nucleotide or nucleotide analog to allow strand elongation to resume.

Typically, after each nucleotide extension step, the reactants are washed away from the solid support prior to the removal of the blocking moiety. Once the blocking moiety has been removed, new reactants are added, allowing the cycle to start anew. At the conclusion of the cycles of extension and deblocking, the finished full-length, single-strand nucleic acid is complete and can be cleaved from the solid support and recovered for subsequent use in applications such as DNA sequencing or PCR. Alternatively, the finished, full-length, single-stranded oligonucleotide can remain attached to the solid support for subsequent use in applications such as hybridization analysis or protein or DNA affinity capture. In other embodiments, partially double-stranded DNA can be used as an initiator, resulting in the synthesis of double-stranded oligonucleotides.

In general, the removal of the blocking moiety depends on the type of blocking moiety used and the chemical bonds by which it is attached to the nucleotide analog. For embodiments in which the blocking moiety is attached to the nucleotide analog via the 3′ carbon of the ribose ring, a variety of removal methods can be used. The 3′-aminoalkoxy group of a nucleotide analog can be converted to a 3′-OH group by removal of the —NH₂ group using sodium nitrite, pH 5.5, at room temperature, as described in Hutter, D., et al. Nucleosides Nucleotides Nucleic Acids, 2010, 29(11): 879-95. The 3′-O-azidomethyl group of a nucleotide analog can be removed by cleavage with tris (2-carboxyethyl) phosphine (TCEP). The 3′-O-cyanoethyl group of a nucleotide analog can be removed, for example, by exposure to 0.25N KOH at 70° C. for 5 minutes. Other options for 3′-modified nucleotide analogs include the use of a palladium catalyst in neutral aqueous solution at elevated temperature hydrochloric acid to pH 2 or a reducing agent such as mercaptoethanol. See, e.g., U.S. Pat. No. 6,664,079; Meng, et al. J. Org. Chem., 2006, 71(81):3248-52; Bi et al., J. Amer. Chem. Soc. 2006; 2542-2543, U.S. Pat. No. 7,279,563, and U.S. Pat. No. 7,414,116, all of which are incorporated herein by reference in their entireties. In other embodiments, the 3′-substitution group may be removed by UV irradiation (see, e.g., WO 92/10587, incorporated by reference herein in its entirety). In some embodiments, the removal of the 3′-O-blocking moiety does not include chemical cleavage but uses a cleaving enzyme such as alkaline phosphatase.

Similarly, in embodiments in which the blocking moiety is attached via a base of the nucleotide analog, the method of removal depends on the nature of the attachment. If a linker-inhibitor blocking moiety as described above is used, removal could occur by chemical, electrochemical, enzymatic, or photolytic cleavage of the linker. For example and without limitation, the linkers can be cleaved by any of the following methods: adjusting the pH of the surrounding environment; adjusting the temperature to change the activity of an enzyme that is activated at a given temperature but inactivated at another temperature; or reduction of disulfide bonds

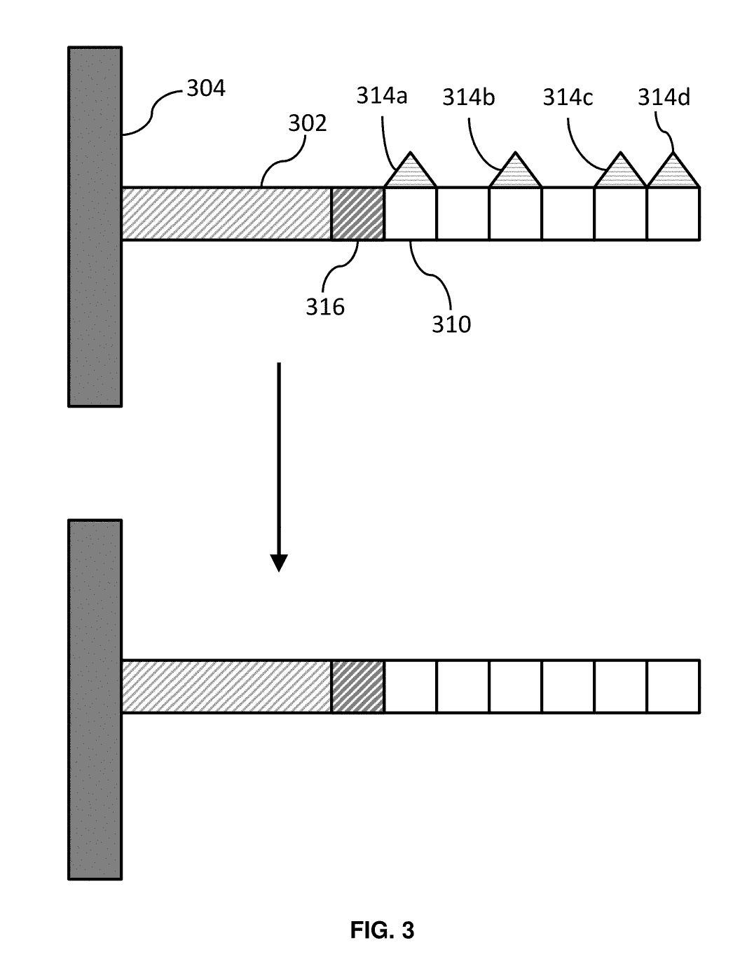

FIG. 3 shows the removal of removable base-pair-inhibiting and/or rate-enhancing moieties 314a, 314b, 314c, and 314d from a nascent oligonucleotide. The following components shown in FIG. 3 are described above in relation to FIG. 1; a solid support 304; an initiator nucleic acid 302, which includes a terminal nucleotide 316, attached to the solid support; and multiple nucleotide analogs (not labeled), some of which have base-pair-inhibiting and/or rate-enhancing moieties 314a, 314b, 314c, and 314d. In the step illustrated in FIG. 3, after elongation of the olignonucleotide strand has been completed, the base-pair-inhibiting and/or rate-enhancing moieties 314a-d are removed from the nucleotide analogs to produce an oligonucleotide capable of annealing to a nucleic acid having a complementary sequence. In the oligonucleotide shown in the illustration, some, but not all, nucleotide analogs have base-pair-inhibiting moieties. However, it is possible within the scope of the invention to have base-pair-inhibiting moieties on all nucleotide analogs of the oligonucleotide or on any percentage of the nucleotide analogs. Generally, an oligonucleotide includes four nucleotide analogs corresponding to the four naturally-occurring nucleotides in DNA or RNA, and in certain embodiments only or two of nucleotide analogs have base-pair-inhibiting moieties. If multiple nucleotide analogs have base-pair-inhibiting moieties, it is advantageous to have similar chemical modifications to the bases of the different modified nucleotide analogs to allow removal of all base-pair-inhibiting moieties in the oligonucleotide in a single reaction. For example, the bases of multiple nucleotide analogs may have acyl modifications on exocyclic amines.

The methods may include a step of cleavage of all or a portion of the initiator nucleic acid from the solid support. The mechanism of cleavage depends on the nature of the attachment between the initiator nucleic acid and solid support and may be achieved chemically, enzymatically, or by any other method known in the art.

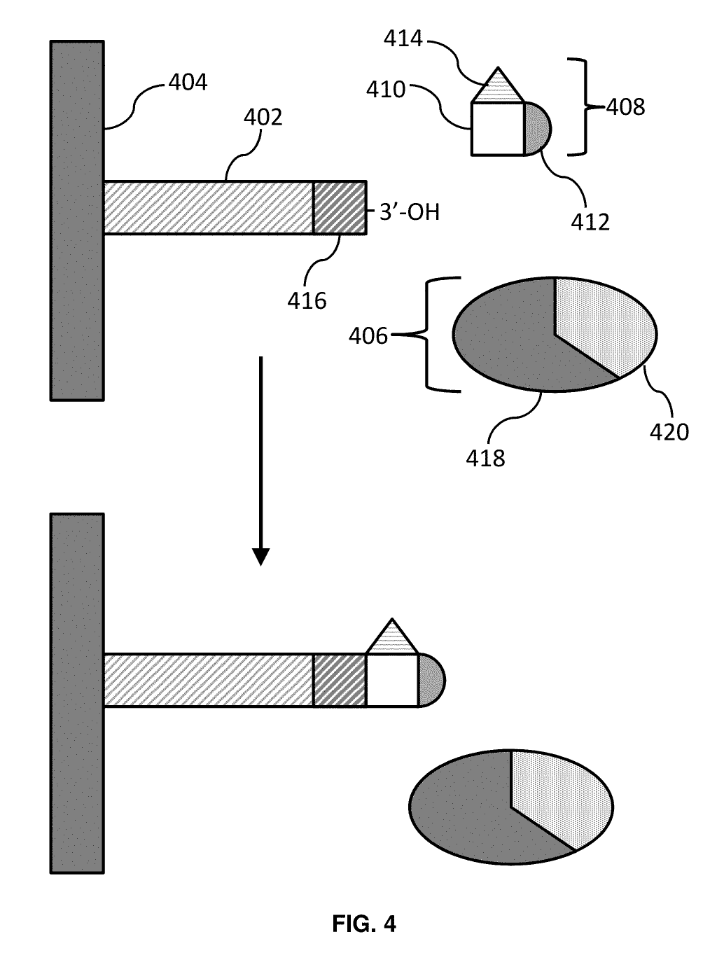

FIG. 4 illustrates a method for template-independent nucleic acid synthesis according to an embodiment of the invention. An initiator nucleic acid 402 bound to a solid substrate 404 is combined with a thermostable DNA polymerase 406 and a nucleotide analog 408 in an aqueous solution. The nucleotide analog 408 includes a nucleotide component 410 and removable blocking moiety 412. The nucleotide analog may also include a removable base-pair-inhibiting and/or rate-enhancing moiety 414, as shown in FIG. 4. The initiator nucleic acid 402 has a terminal nucleotide 416 with a free 3′-OH group. The thermostable DNA polymerase 406 catalyzes the formation of a covalent bond between the nucleotide analog 408 and the terminal nucleotide 416 of the initiator nucleic acid 402. The presence of the blocking moiety 412 on the nucleotide analog 408 prevents strand elongation by blocking the thermostable DNA polymerase 406 from attaching additional nucleotides (not shown) or nucleotide analogs 408.

The thermostable DNA polymerase 406 may be a thermostable DNA polymerase polypeptide. As used herein, a “thermostable DNA polymerase polypeptide” refers to any polypeptide that has DNA polymerase activity at elevated temperatures, e.g., >42° C. The thermostable DNA polymerase polypeptide may be from a naturally-occurring thermostable DNA polymerase, or it may be a non-naturally-occurring polypeptide that includes one or more amino acid sequences derived from a naturally-occurring thermostable DNA polymerase and one or more amino acid sequences derived from a DNA polymerase theta of any organism. For example, the thermostable polymerase 406 may include a thermostable polymerase domain 418 that promotes activity at high temperatures and a DNA polymerase theta domain 420 that promotes template-independent synthesis. Preferably, the source of a naturally-occurring thermostable DNA polymerase is an A-family DNA polymerase, such as Taq polymerase. The thermostable DNA polymerase polypeptide may have one or more amino acid sequences identical to those in a naturally-occurring thermostable DNA polymerase. The thermostable DNA polymerase polypeptide may have one or more amino acid sequences that have alterations to amino acid sequences from a naturally-occurring thermostable DNA polymerase. The thermostable DNA polymerase polypeptide may have one or more amino acid sequences identical to those in a naturally-occurring DNA polymerase theta. The thermostable DNA polymerase polypeptide may have one or more amino acid sequences that have alterations to amino acid sequences from a naturally-occurring DNA polymerase theta. The alterations may include amino acid substitutions, insertions, deletions, or modifications. The thermostable DNA polymerase polypeptide may have an amino acid sequence that is at least 70%, at least 75%, at least 80%, at least 85%, at least 90%, at least 95%, at least 96%, at least 97%, at least 98%, or at least 99% identical to one of SEQ ID NOS:5-7.

An advantage of using a thermostable DNA polymerase is that the elongation reaction can be performed at temperatures that minimize or prevent self-annealing of the nascent oligonucleotide. Because the nascent oligonucleotide does not form hairpin structures that hinder the rate and/or yield of enzymatic extension, the efficiency of synthesis of the desired full-length product is improved. In addition, the use of a thermostable DNA polymerase at elevated temperatures obviates the need for nucleotide analogs that have base-pair-inhibiting moieties and eliminates the requirement for a step in which such structures are removed from the oligonucleotide. The temperature at which the elongation reaction can occur depends on the stability of the thermostable DNA polymerase. The elongation reaction may be performed at temperatures >42° C., >45° C., >50° C., >55° C., >60° C., >65° C., >70° C., or >75° C.

The aqueous solution may contain Mn²⁺. The Mn²⁺ concentration may be about 0.05 mM, about 0.1 mM, about 0.2 mM, about 0.5 mM, about 1 mM, about 2 mM, about 5 mM, or about 10 mM.

For methods of oligonucleotide synthesis using a thermostable DNA polymerase 406, it is understood that the elongation reaction is followed by a deblocking reaction, as described above for synthesis methods using DNA polymerase theta. Because the elongation and deblocking steps occur sequentially, the deblocking step may, but need not, occur at elevated temperatures as well.

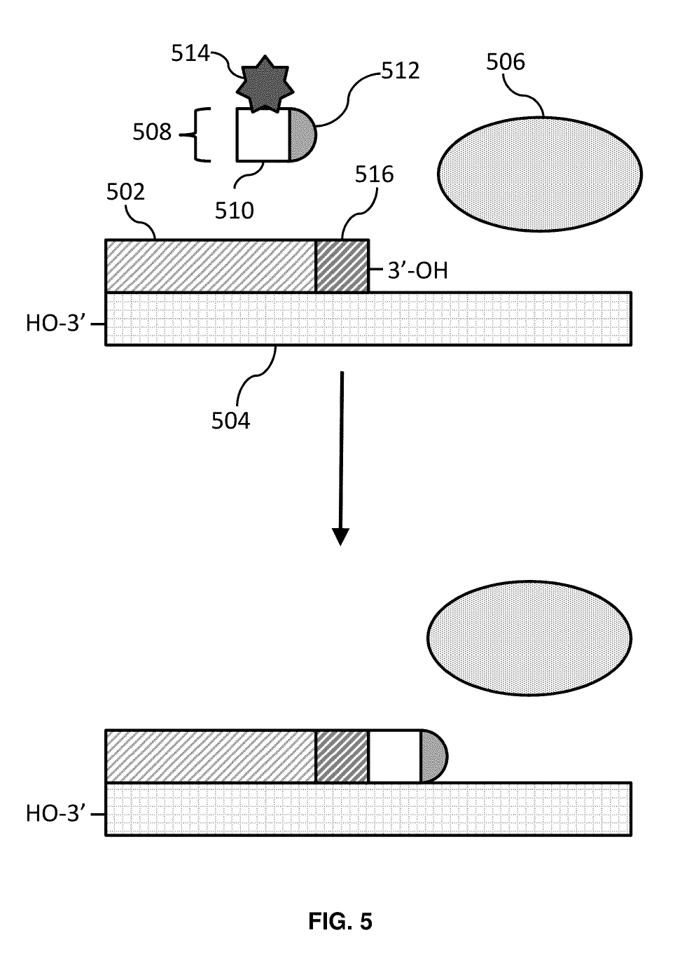

FIG. 5 illustrates a method for template-directed nucleic acid synthesis of according to an embodiment of the invention. A nucleic acid primer 502, which includes a 3′ terminal nucleotide 516, is annealed to a complementary sequence in a nucleic acid template 504 in an aqueous solution containing a DNA polymerase theta 506 and one or more 3′-aminoalkoxy nucleotide analogs 508. The 3′-aminoalkoxy nucleotide analog 508 includes a nucleotide component 510 and a 3′-NH₂ group 512. The 3′-aminoalkoxy nucleotide analog may also include a removable label 514, as shown in FIG. 5. The DNA polymerase theta 506 catalyzes the formation of a covalent bond between the nucleotide analog 508 and the terminal nucleotide 516 of the nucleic acid primer 502. The presence of the 3′-NH₂ group 512 on the nucleotide analog 508 prevents strand elongation by blocking the DNA polymerase 506 from attaching additional nucleotides (not shown) or 3′-aminoalkoxy nucleotide analogs 508.

Preferably, the aqueous solution contains Mg²⁺. The Mg²⁺ concentration may be about 0.05 mM, about 0.1 mM, about 0.2 mM, about 0.5 mM, about 1 mM, about 2 mM, about 5 mM, or about 10 mM.

The removable label 514 may be any detectable moiety. For example, the label may detectable by fluorescence, luminescence, radiography, spectroscopy, or other methods known in the art. Preferably, the label is fluorescent. Nucleotide analogs having removable labels and methods of detecting and removing labels are known in the art.

The nucleotide analog 508 may be one of a set that corresponds to the four naturally-occurring nucleotides in DNA or RNA and in which each nucleotide analog has a unique label that enables the identification of the base in that particular analog by detecting the label. Alternatively, one nucleotide analog in the set may lack a label, and its base can be identified by the lack of signal, in contrast to the signals produced by the other three nucleotide analogs. In a variation of this embodiment, only two labels are used among the three labeled nucleotide analogs, with two labeled nucleotides having a single label and the third labeled nucleotide having a combination of the two labels. In this embodiment, the doubly-labeled nucleotide is identifiable by a signal given from the combination of labels that is different from the signal provided by either label individually.

The nucleic acid primer 502, the nucleic acid template 504, or both may be bound to a solid support, as described above in relation to methods for template-independent oligonucleotide synthesis. Although the free 3′-OH end of the nucleic acid template 504 is shown for reference, the nucleic acid template 504 may be bound to a solid support at this end of the molecule and thus may not have a free —OH group at its 3′ end.

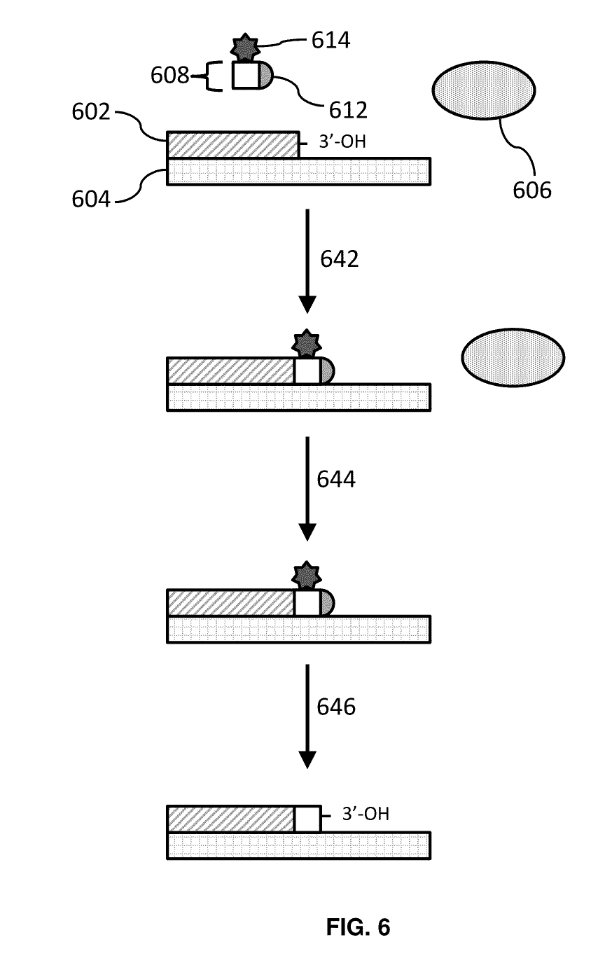

FIG. 6 illustrates a method for determining the nucleotide sequence of a nucleic acid according to an embodiment of the invention. A template 604 from a strand of the nucleic acid to be sequences is annealed to a nucleic acid primer 602 that is complementary to a sequence in the template 604 in an aqueous solution that includes a DNA polymerase theta 606 and a set of 3′-aminoalkoxy nucleotide analogs 608. The 3′-aminoalkoxy nucleotide triphosphate analogs include removable labels 614. In the first step 642, DNA polymerase theta 606 catalyzes the addition to the primer 602 of a labeled 3′-aminoalkoxy nucleotide analog 608 that is complementary to the template nucleotide immediately 5′ of the sequence complementary to the primer 602. In the second step 644, the label of the newly-added 3′-aminoalkoxy nucleotide analog 608 is detected. In the third step 646, the label 614 and 3′-NH₂ group 612 are removed from the 3′-aminoalkoxy nucleotide analog 608. The label 614 and 3′-NH₂ group 612 may be removed from the 3′-aminoalkoxy nucleotide analog 608 simultaneously or sequentially in either order. This series of steps is repeated for as many cycles as needed to reach the 5′ end of the template stand 604. Thus, the nucleotide sequence of the synthesized strand is measured directly, and the nucleotide sequence of the template is inferred due to its complementarity.

Preferably, the aqueous solution in the first step contains Mg²⁺. The Mg²⁺ concentration may be about 0.05 mM, about 0.1 mM, about 0.2 mM, about 0.5 mM, about 1 mM, about 2 mM, about 5 mM, or about 10 mM.

The nucleic acid primer 602, the 604, or both may be bound to a solid support, as described above in relation to methods for template-independent oligonucleotide synthesis.

It will be understood that the reaction conditions may not be identical for the different steps in the methods for determining a nucleotide sequence of a nucleic acid molecule. Thus, the methods may include intermediate washing steps.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### SEQUENCES

SEQ ID NO: 1
MNLLRRSGKRRRSESGSDSFSGSGGDSSASPQFLSGSVLSPPPGLGR
CLKAAAAGECKPTVPDYERDKLLLANWGLPKAVLEKYHSFGVKKMFE
WQAECLLLGQVLEGKNLVYSAPTSAGKTLVAELLILKRVLEMRKKAL
FILPFVSVAKEKKYYLQSLFQEVGIKVDGYMGSTSPSRHFSSLDIAV
CTIERANGLINRLIEENKMDLLGMVVVDELHMLGDSHRGYLLELLLT
KICYITRKSASCQADLASSLSNAVQIVGMSATLPNLELVASWLNAEL
YHTDFRPVPLLESVKVGNSIYDSSMKLVREFEPMLQVKGDEDHVVSL
CYETICDNHSVLLFCPSKKWCEKLADIIAREFYNLHHQAEGLVKPSE
CPPVILEQKELLEVMDQLRRLPSGLDSVLQKTVPWGVAFHHAGLTFE
ERDIIEGAFRQGLIRVLAATSTLSSGVNLPARRVIIRTPIFGGRPLD
ILTYKQMVGRAGRKGVDTVGESILICKNSEKSKGIALLQGSLKPVRS
CLQRREGEEVTGSMIRAILEIIVGGVASTSQDMHTYAACTFLAASMK
EGKQGIQRNQESVQLGAIEACVMWLLENEFIQSTEASDGTEGKVYHP
THLGSATLSSSLSPADTLDIFADLQRAMKGFVLENDLHILYLVTPMF
EDWTTIDWYRFFCLWEKLPTSMKRVAELVGVEEGFLARCVKGKVVAR
TERQHRQMAIHKRFFTSLVLLDLISEVPLREINQKYGCNRGQIQSLQ
QSAAVYAGMITVFSNRLGWHNMELLLSQFQKRLTFGIQRELCDLVRV
SLLNAQRARVLYASGFHTVADLARANIVEVEVILKNAVPFKSARKAV
DEEEEAVEERRNMRTIWVTGRKGLTEREAAALIVEEARMILQQDLVE
MGVQWNPCALLHSSTCSLTHSESEVKEHTFISQTKSSYKKLTSKNKS
NTIFSDSYIKHSPNIVQDLNKSREHTSSFNCNFQNGNQEHQTCSIFR
ARKRASLDINKEKPGASQNEGKTSDKKVVQTFSQKTKKAPLNFNSEK
MSRSFRSWKRRKHLKRSRDSSPLKDSGACRIHLQGQTLSNPSLCEDP
FTLDEKKTEFRNSGPFAKNVSLSGKEKDNKTSFPLQIKQNCSWNITL
TNDNFVEHIVTGSQSKNVTCQATSVVSEKGRGVAVEAEKINEVLIQN
GSKNQNVYMKHHDIHPINQYLRKQSHEQTSTITKQKNIIERQMPCEA
VSSYINRDSNVTINCERIKLNTEENKPSHFQALGDDISRTVIPSEVL
PSAGAFSKSEGQHENFLNISRLQEKTGTYTTNKTKNNHVSDLGLVLC
DFEDSFYLDTQSEKIIQQMATENAKLGAKDTNLAAGIMQKSLVQQNS
MNSFQKECHIPFPAEQHPLGATKIDHLDLKTVGTMKQSSDSHGVDIL
TPESPIFHSPILLEENGLFLKKNEVSVTDSQLNSFLQGYQTQETVKP
VILLIPQKRTPTGVEGECLPVPETSLNMSDSLLFDSFSDDYLVKEQL
PDMQMKEPLPSEVTSNHFSDSLCLQEDLIKKSNVNENQDTHQQLTCS
NDESIIFSEMDSVQMVEALDNVDIFPVQEKNHTVVSPRALELSDPVL
DEHHQGDQDGGDQDERAEKSKLTGTRQNHSFIWSGASFDLSPGLQRI
LDKVSSPLENEKLKSMTINFSSLNRKNTELNEEQEVISNLETKQVQG
ISFSSNNEVKSKIEMLENNANHDETSSLLPRKESNIVDDNGLIPPTP
IPTSASKLTFPGILETPVNPWKTNNVLQPGESYLFGSPSDIKNHDLS
PGSRNGFKDNSPISDTSFSLQLSQDGLQLTPASSSSESLSIIDVASD
QNLFQTFIKEWRCKKRFSISLACEKIRSLTSSKTATIGSRFKQASSP
QEIPIRDDGFPIKGCDDTLVVGLAVCWGGRDAYYFSLQKEQKHSEIS
ASLVPPSLDPSLTLKDRMWYLQSCLRKESDKECSVVIYDFIQSYKIL
LLSCGISLEQSYEDPKVACWLLDPDSQEPTLHSIVTSFLPHELPLLE
GMETSQGIQSLGLNAGSEHSGRYRASVESILIFNSMNQLNSLLQKEN
LQDVFRKVEMPSQYCLALLELNGIGFSTAECESQKHIMQAKLDAIET
QAYQLAGHSFSFTSSDDIAEVLFLELKLPPNREMKNQGSKKTLGSTR
RGIDNGRKLRLGRQFSTSKDVLNKLKALHPLPGLILEWRRITNAITK
VVFPLQREKCLNPFLGMERIYPVSQSHTATGRITFTEPNIQNVPRDF
EIKMPTLVGESPPSQAVGKGLLPMGRGKYKKGFSVNPRCQAQMEERA
ADRGMPFSISMRHAFVPFPGGSILAADYSQLELRILAHLSHDRRLIQ
VLNTGADVFRSIAAEWKMIEPESVGDDLRQQAKQICYGIIYGMGAKS
LGEQMGIKENDAACYIDSFKSRYTGINQFMTETVKNCKRDGFVQTIL
GRRRYLPGIKDNNPYRKAHAERQAINTIVQGSAADIVKIATVNIQKQ
LETFHSTFKSHGHREGMLQSDQTGLSRKRKLQGMFCPIRGGFFILQL
HDELLYEVAEEDVVQVAQIVKNEMESAVKLSVKLKVKVKIGASWGEL
KDFDV
SEQ ID NO: 2
MNLLRRSGKRRRSESGSDSFSGSGGDSSASPQFLSGSVLSPPPGLGR
CLKAAAAGECKPTVPDYERDKLLLANWGLPKAVLEKYHSFGVKKMFE
WQAECLLLGQVLEGKNLVYSAPTSAGKTLVAELLILKRVLEMRKKAL
FILPFVSVAKEKKYYLQSLFQEVGIKVDGYMGSTSPSRHFSSLDIAV
CTIERANGLINRLIEENKMDLLGMVVVDELHMLGDSHRGYLLELLLT
KICYITRKSASCQADLASSLSNAVQIVGMSATLPNLELVASWLNAEL
YHTDFRPVPLLESVKVGNSIYDSSMKLVREFEPMLQVKGDEDHVVSL
CYETICDNHSVLLFCPSKKWCEKLADIIAREFYNLHHQAEGLVKPSE
CPPVILEQKELLEVMDQLRRLPSGLDSVLQKTVPWGVAFHHAGLTFE
ERDIIEGAFRQGLIRVLAATSTLSSGVNLPARRVIIRTPIFGGRPLD
ILTYKQMVGRAGRKGVDTVGESILICKNSEKSKGIALLQGSLKPVRS
CLQRREGEEVTGSMIRAILEIIVGGVASTSQDMHTYAACTFLAASMK
EGKQGIQRNQESVQLGAIEACVMWLLENEFIQSTEASDGTEGKVYHP
THLGSATLSSSLSPADTLDIFADLQRAMKGFVLENDLHILYLVTPMF
EDWTTIDWYRFFCLWEKLPTSMKRVAELVGVEEGFLARCVKGKVVAR
TERQHRQMAIHKRFFTSLVLLDLISEVPLREINQKYGCNRGQIQSLQ
QSAAVYAGMITVFSNRLGWHNMELLLSQFQKRLTFGIQRELCDLVRV
SLLNAQRARVLYASGFHTVADLARANIVEVEVILKNAVPFKSARKAV
DEEEEAVEERRNMRTIWVTGRKGLTEREAAALIVEEARMILQQDLVE
M
SEQ ID NO: 3
GVQWNPCALLHSSTCSLTHSESEVKEHTFISQTKSSYKKLTSKNKSN
TIFSDSYIKHSPNIVQDLNKSREHTSSFNCNFQNGNQEHQTCSIFRA
RKRASLDINKEKPGASQNEGKTSDKKVVQTFSQKTKKAPLNFNSEKM
SRSFRSWKRRKHLKRSRDSSPLKDSGACRIHLQGQTLSNPSLCEDPF
TLDEKKTEFRNSGPFAKNVSLSGKEKDNKTSFPLQIKQNCSWNITLT
NDNFVEHIVTGSQSKNVTCQATSVVSEKGRGVAVEAEKINEVLIQNG
SKNQNVYMKHHDIHPINQYLRKQSHEQTSTITKQKNIIERQMPCEAV
SSYINRDSNVTINCERIKLNTEENKPSHFQALGDDISRTVIPSEVLP
SAGAFSKSEGQHENFLNISRLQEKTGTYTTNKTKNNHVSDLGLVLCD
FEDSFYLDTQSEKIIQQMATENAKLGAKDTNLAAGIMQKSLVQQNSM
NSFQKECHIPFPAEQHPLGATKIDHLDLKTVGTMKQSSDSHGVDILT
PESPIFHSPILLEENGLFLKKNEVSVTDSQLNSFLQGYQTQETVKPV
ILLIPQKRTPTGVEGECLPVPETSLNMSDSLLFDSFSDDYLVKEQLP
DMQMKEPLPSEVTSNHFSDSLCLQEDLIKKSNVNENQDTHQQLTCSN
DESIIFSEMDSVQMVEALDNVDIFPVQEKNHTVVSPRALELSDPVLD
EHHQGDQDGGDQDERAEKSKLTGTRQNHSFIWSGASFDLSPGLQRIL
DKVSSPLENEKLKSMTINFSSLNRKNTELNEEQEVISNLETKQVQGI
SFSSNNEVKSKIEMLENNANHDETSSLLPRKESNIVDDNGLIPPTPI
PTSASKLTFPGILETPVNPWKTNNVLQPGESYLFGSPSDIKNHDLSP
GSRN
SEQ ID NO: 4
GFKDNSPISDTSFSLQLSQDGLQLTPASSSSESLSIIDVASDQNLFQ
TFIKEWRCKKRFSISLACEKIRSLTSSKTATIGSRFKQASSPQEIPI
RDDGFPIKGCDDTLVVGLAVCWGGRDAYYFSLQKEQKHSEISASLVP
PSLDPSLTLKDRMWYLQSCLRKESDKECSVVIYDFIQSYKILLLSCG
ISLEQSYEDPKVACWLLDPDSQEPTLHSIVTSFLPHELPLLEGMETS
QGIQSLGLNAGSEHSGRYRASVESILIFNSMNQLNSLLQKENLQDVF
RKVEMPSQYCLALLELNGIGFSTAECESQKHIMQAKLDAIETQAYQL
AGHSFSFTSSDDIAEVLFLELKLPPNREMKNQGSKKTLGSTRRGIDN
GRKLRLGRQFSTSKDVLNKLKALHPLPGLILEWRRITNAITKVVFPL
QREKCLNPFLGMERIYPVSQSHTATGRITFTEPNIQNVPRDFEIKMP
TLVGESPPSQAVGKGLLPMGRGKYKKGFSVNPRCQAQMEERAADRGM
PFSISMRHAFVPFPGGSILAADYSQLELRILAHLSHDRRLIQVLNTG
ADVFRSIAAEWKMIEPESVGDDLRQQAKQICYGIIYGMGAKSLGEQM
GIKENDAACYIDSFKSRYTGINQFMTETVKNCKRDGFVQTILGRRRY
LPGIKDNNPYRKAHAERQAINTIVQGSAADIVKIATVNIQKQLETFH
STFKSHGHREGMLQSDQTGLSRKRKLQGMFCPIRGGFFILQLHDELL
YEVAEEDVVQVAQIVKNEMESAVKLSVKLKVKVKIGASWGELKDFDV
SEQ ID NO: 5
MKNQGSKKTLGSTRRGIDNGRK
SEQ ID NO: 6
VGESPPSQAVGKGLLPMGRGKYKKGFSVNPRCQAQMEERAADRGMPF
SISMR
SEQ ID NO: 7
STFKSHGHREGMLQSDQTGLSRKRKLQGMFCPI

What is claimed is:

1. A method for oligonucleotide synthesis, the method comprising
exposing a nucleic acid attached to a solid support in the absence of a nucleic acid template to a nucleotide analog comprising a removable blocking moiety and a DNA polymerase theta polypeptide;
wherein the DNA polymerase theta catalyzes addition of a first nucleotide analog to said nucleic acid but is prevented from catalyzing addition of a subsequent nucleotide analog until said blocking moiety is removed.

2. The method of claim 1, wherein the DNA polymerase theta polypeptide comprises an amino acid sequence at least about 90% identical to SEQ ID NO:4.

3. The method of claim 1, wherein said exposing step is conducted in an aqueous medium comprising Mn²⁺.

4. The method of claim 1, wherein the removable blocking moiety is linked to a 3′ oxygen in a ribose ring of the nucleotide analog.

5. The method of claim 4, wherein the removable blocking moiety comprises a 3′-aminoalkoxy group, a 3′-O-cyanoethyl group or a 3′-O-azidomethyl group

6. The method of claim 1, wherein the removable blocking moiety is linked to a base in the nucleotide analog.

7. The method of claim 6, wherein the removable blocking moiety is linked via N4 of cytosine, N3 of thymine, O4 of thymine, N2 of guanine, O6 of guanine, N6 of adenine, N3 of uracil, or O4 of uracil.

8. The method of claim 1, wherein the nucleotide analog is selected from the group consisting of 3′-aminoalkoxy-N4-acyl-dCTP, 3′-aminoalkoxy-N4-acyl-rCTP, 3′-aminoalkoxy-N2-acyl-dGTP, and 3′-aminoalkoxy-N2-acyl-rGTP.

9. The method of claim 1, further comprising removing the blocking moiety from the first nucleotide analog.

10. The method of claim 1, wherein said solid support is a bead or a well.

11. The method of claim 1, wherein the nucleotide analog further comprises a removable base-pair-inhibiting moiety that:
prevents the nucleotide analog from forming a base pair with another nucleotide or nucleotide analog; and
remains attached to the nucleotide under conditions that result in removal of the removable blocking moiety.

12. The method of claim 11, wherein the removable base-pair-inhibiting moiety is linked to the nucleotide analog via N6 of adenine, N2 of guanine, or N4 of cytosine.

13. The method of claim 11, further comprising:
removing the base-pair-inhibiting moiety from the nucleotide analog.

14. The method of claim 1, wherein the nucleotide analog further comprises a removable rate-enhancing moiety that:
increases the rate of addition of the first nucleotide analog to said nucleic acid by the DNA polymerase theta; and
remains attached to the nucleotide under conditions that result in removal of the removable blocking moiety.

15. The method of claim 14, wherein the removable moiety is linked to the nucleotide analog via N6 of adenine, N2 of guanine, or O6 of guanine.

16. The method of claim 14, further comprising:
removing the rate-enhancing moiety from the nucleotide analog.

17. The method of claim 1, wherein the oligonucleotide is DNA and the nucleotide analog is a 2′-deoxyribonucleotide analog.

18. The method of claim 1, wherein the nucleic acid is DNA and the nucleotide analog is a ribonucleotide analog.

19. The method of claim 1, wherein said Polymerase theta is thermostable.


SEQUENCE LISTING (7 sequences)

SEQ ID NO: 1 - protein, 2590 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEMGVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRNGFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 2 - protein, 894 aa, Homo sapiens
MNLLRRSGKRRRSESGSDSFSGSGGDSSASPQFLSGSVLSPPPGLGRCLKAAAAGECKPTVPDYERDKLLLANWGLPKAVLEKYHSFGVKKMFEWQAECLLLGQVLEGKNLVYSAPTSAGKTLVAELLILKRVLEMRKKALFILPFVSVAKEKKYYLQSLFQEVGIKVDGYMGSTSPSRHFSSLDIAVCTIERANGLINRLIEENKMDLLGMVVVDELHMLGDSHRGYLLELLLTKICYITRKSASCQADLASSLSNAVQIVGMSATLPNLELVASWLNAELYHTDFRPVPLLESVKVGNSIYDSSMKLVREFEPMLQVKGDEDHVVSLCYETICDNHSVLLFCPSKKWCEKLADIIAREFYNLHHQAEGLVKPSECPPVILEQKELLEVMDQLRRLPSGLDSVLQKTVPWGVAFHHAGLTFEERDIIEGAFRQGLIRVLAATSTLSSGVNLPARRVIIRTPIFGGRPLDILTYKQMVGRAGRKGVDTVGESILICKNSEKSKGIALLQGSLKPVRSCLQRREGEEVTGSMIRAILEIIVGGVASTSQDMHTYAACTFLAASMKEGKQGIQRNQESVQLGAIEACVMWLLENEFIQSTEASDGTEGKVYHPTHLGSATLSSSLSPADTLDIFADLQRAMKGFVLENDLHILYLVTPMFEDWTTIDWYRFFCLWEKLPTSMKRVAELVGVEEGFLARCVKGKVVARTERQHRQMAIHKRFFTSLVLLDLISEVPLREINQKYGCNRGQIQSLQQSAAVYAGMITVFSNRLGWHNMELLLSQFQKRLTFGIQRELCDLVRVSLLNAQRARVLYASGFHTVADLARANIVEVEVILKNAVPFKSARKAVDEEEEAVEERRNMRTIWVTGRKGLTEREAAALIVEEARMILQQDLVEM

SEQ ID NO: 3 - protein, 897 aa, Homo sapiens
GVQWNPCALLHSSTCSLTHSESEVKEHTFISQTKSSYKKLTSKNKSNTIFSDSYIKHSPNIVQDLNKSREHTSSFNCNFQNGNQEHQTCSIFRARKRASLDINKEKPGASQNEGKTSDKKVVQTFSQKTKKAPLNFNSEKMSRSFRSWKRRKHLKRSRDSSPLKDSGACRIHLQGQTLSNPSLCEDPFTLDEKKTEFRNSGPFAKNVSLSGKEKDNKTSFPLQIKQNCSWNITLTNDNFVEHIVTGSQSKNVTCQATSVVSEKGRGVAVEAEKINEVLIQNGSKNQNVYMKHHDIHPINQYLRKQSHEQTSTITKQKNIIERQMPCEAVSSYINRDSNVTINCERIKLNTEENKPSHFQALGDDISRTVIPSEVLPSAGAFSKSEGQHENFLNISRLQEKTGTYTTNKTKNNHVSDLGLVLCDFEDSFYLDTQSEKIIQQMATENAKLGAKDTNLAAGIMQKSLVQQNSMNSFQKECHIPFPAEQHPLGATKIDHLDLKTVGTMKQSSDSHGVDILTPESPIFHSPILLEENGLFLKKNEVSVTDSQLNSFLQGYQTQETVKPVILLIPQKRTPTGVEGECLPVPETSLNMSDSLLFDSFSDDYLVKEQLPDMQMKEPLPSEVTSNHFSDSLCLQEDLIKKSNVNENQDTHQQLTCSNDESIIFSEMDSVQMVEALDNVDIFPVQEKNHTVVSPRALELSDPVLDEHHQGDQDGGDQDERAEKSKLTGTRQNHSFIWSGASFDLSPGLQRILDKVSSPLENEKLKSMTINFSSLNRKNTELNEEQEVISNLETKQVQGISFSSNNEVKSKIEMLENNANHDETSSLLPRKESNIVDDNGLIPPTPIPTSASKLTFPGILETPVNPWKTNNVLQPGESYLFGSPSDIKNHDLSPGSRN

SEQ ID NO: 4 - protein, 799 aa, Homo sapiens
GFKDNSPISDTSFSLQLSQDGLQLTPASSSSESLSIIDVASDQNLFQTFIKEWRCKKRFSISLACEKIRSLTSSKTATIGSRFKQASSPQEIPIRDDGFPIKGCDDTLVVGLAVCWGGRDAYYFSLQKEQKHSEISASLVPPSLDPSLTLKDRMWYLQSCLRKESDKECSVVIYDFIQSYKILLLSCGISLEQSYEDPKVACWLLDPDSQEPTLHSIVTSFLPHELPLLEGMETSQGIQSLGLNAGSEHSGRYRASVESILIFNSMNQLNSLLQKENLQDVFRKVEMPSQYCLALLELNGIGFSTAECESQKHIMQAKLDAIETQAYQLAGHSFSFTSSDDIAEVLFLELKLPPNREMKNQGSKKTLGSTRRGIDNGRKLRLGRQFSTSKDVLNKLKALHPLPGLILEWRRITNAITKVVFPLQREKCLNPFLGMERIYPVSQSHTATGRITFTEPNIQNVPRDFEIKMPTLVGESPPSQAVGKGLLPMGRGKYKKGFSVNPRCQAQMEERAADRGMPFSISMRHAFVPFPGGSILAADYSQLELRILAHLSHDRRLIQVLNTGADVFRSIAAEWKMIEPESVGDDLRQQAKQICYGIIYGMGAKSLGEQMGIKENDAACYIDSFKSRYTGINQFMTETVKNCKRDGFVQTILGRRRYLPGIKDNNPYRKAHAERQAINTIVQGSAADIVKIATVNIQKQLETFHSTFKSHGHREGMLQSDQTGLSRKRKLQGMFCPIRGGFFILQLHDELLYEVAEEDVVQVAQIVKNEMESAVKLSVKLKVKVKIGASWGELKDFDV

SEQ ID NO: 5 - protein, 22 aa, Homo sapiens
MKNQGSKKTLGSTRRGIDNGRK

SEQ ID NO: 6 - protein, 52 aa, Homo sapiens
VGESPPSQAVGKGLLPMGRGKYKKGFSVNPRCQAQMEERAADRGMPFSISMR

SEQ ID NO: 7 - protein, 33 aa, Homo sapiens
STFKSHGHREGMLQSDQTGLSRKRKLQGMFCPI